(19) United States
(12) Patent Application Publication
(10) Pub. No.: US 2008/0214535 A1
(43) Pub. Date: Sep. 4, 2008

(54) Amorphous Aprepitant Coprecipitates

(75) Inventors: Vijayavitthal Thippannachar Mathad, Hyderabad. (IN); Pravinchandra Vankawala Jayantilal, Surat (IN); Chandrasekhar Ravi Ram Elati, Visakhapatnam (IN); Subrahmanyeswara Rao Chlamala, Krishna District. (IN); Naveen Kumar Kolla, Prakasam District (IN); Srinivas Gangula, Karimnagar District (IN); Srirami Reddy Kikkuru, Guntur (IN); Srinivasan Neti, Bhimavaram (IN); Raveendra Reddy Chinta, Chittoor (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignees: DR. REDDY'S LABORATORIES LTD., Hyderabad, AP (IN); DR. REDDY'S LABORATORIES, INC., Bridgewater, NJ (US)

(21) Appl. No.: 11/997,203

(22) PCT Filed: Jul. 31, 2006
(86) PCT No.: PCT/US06/29973

§ 371 Date: Jan. 29, 2008

Related U.S. Application Data
(60) Provisional application No. 60/703,600, filed on Jul. 29, 2005.

(51) Int. Cl. A61K 31/5377

(52) U.S. Cl. 514/230.8

(57) ABSTRACT

A coprecipitate comprising amorphous aprepitant and a pharmaceutically acceptable carrier is prepared by rapidly removing solvent from a solution containing aprepitant and the carrier.

### INTRODUCTION TO THE INVENTION

The present invention relates to coprecipitates comprising aprepitant. More specifically, the invention relates to coprecipitates comprising aprepitant with improved physicochemical characteristics which help in the effective delivery of aprepitant.

Aprepitant has the chemical name 5-[[(2R,3S)-2-[(1R)-1-[3,5-bis(trifluoromethyl)phenyl]ethoxy]-3-(4-fluorophenyl)-4-morpholinyl]methyl]-1,2-dihydro-3H-1,2,4-triazol-3-one, and is structurally represented by Formula I.

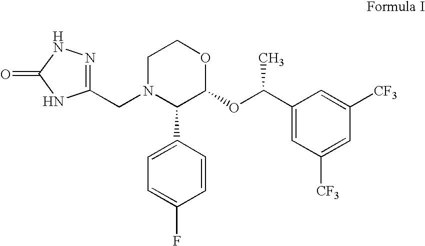

Aprepitant is a NK1 receptor antagonist, useful as an antiemetic agent. It is approved for the treatment of emesis associated with chemotherapy and is commercially available in the United States as EMEND™ capsules containing 80 mg or 125 mg of aprepitant for oral administration.

U.S. Pat. Nos. 6,096,742 and 6,583,142 describe two polymorphic forms of aprepitant, viz. Form I and Form II. Form I is said to be thermodynamically more stable than Form II based on its lower solubility.

Aprepitant is practically insoluble in water, sparingly soluble in ethanol and isopropyl acetate and slightly soluble in acetonitrile. Aprepitant is a molecule having poor solubility and poor permeability characteristics. Additionally, the delivery of aprepitant is also fraught with high inter-patient variability when delivered as a tablet formulation, thereby requiring a nanoparticulate composition to overcome this problem. The poor solubility of aprepitant in aqueous media and poor delivery characteristics pose a tremendous challenge to the pharmaceutical formulation scientist in providing for its delivery in adequate concentrations into the systemic circulation.

International Application Publication No. WO 03/049718 describes nanoparticulate (<2000 nm) particles of aprepitant, having an adsorbed discrete phase surface stabilizer. The surface stabilizer is described as physically adhering to aprepitant particle surfaces but not chemically bonded to, or reacted with, the aprepitant.

The rate of dissolution of a poorly water-soluble drug is a rate-limiting factor in its absorption by the body. It is generally known that a reduction in the particle size of an active ingredient can result in an increase in the dissolution rate of such compounds through an increase in the surface area of the solid phase that comes in contact with the aqueous medium. Different active ingredients demonstrate an enhancement in dissolution rate to different extents. There is no way to predict the extent to which the dissolution rate of an active will be enhanced through particle size reduction or what is the desired particle size for achieving the desired bioavailability characteristics.

Particle size reduction beyond certain stage may many times result in other material handling and processing issues such as generation of static charges on new exposed surfaces and agglomeration thereby resulting in unpredictable variations in solubility, dissolution and hence bioavailability. Such problem is addressed in the art by using surface stabilizers to prevent agglomeration.

There is thus a long-felt need for the development of pharmaceutical compositions of aprepitant with improved solubility properties and improved bioavailability characteristics. This would be a significant improvement in the field of pharmaceutical science.

### SUMMARY OF THE INVENTION

The present invention relates to coprecipitates of amorphous aprepitant and a pharmaceutically acceptable carrier, having improved physicochemical characteristics that assist in the effective delivery of aprepitant.

In an aspect, the invention provides a process for the preparation of coprecipitates of amorphous aprepitant and a pharmaceutically acceptable carrier.

In an aspect, a process for preparation of coprecipitates of amorphous aprepitant with a pharmaceutically acceptable carrier comprises the steps of:

a) providing a solution of aprepitant and a pharmaceutically acceptable carrier;

b) removing the solvent; and

c) optionally, drying the solid that is obtained.

Powder compositions of the invention have improved solubility properties and hence also have improved bioavailability.

### BRIEF DESCRIPTION OF THE DRAWINGS

FIG. 1 is an X-ray powder diffraction (“XRD”) pattern of amorphous aprepitant, prepared in Control Example 2.

FIG. 2 is an XRD pattern of a coprecipitate of an amorphous aprepitant coprecipitate with povidone in a weight ratio of 1:1 prepared in Example 1.

FIG. 3 is an XRD pattern of a coprecipitate of an amorphous aprepitant coprecipitate with povidone in a weight ratio of 1:1 prepared in Example 2.

FIG. 4 is an XRD pattern of an amorphous aprepitant coprecipitate with povidone in a weight ratio of 3:1 prepared in Example 3.

FIG. 5 is an infrared absorption spectrum of an amorphous aprepitant coprecipitate with povidone in a weight ratio of 1:1 prepared in Example 6.

FIG. 6 is a differential scanning calorimetric curve of an amorphous aprepitant coprecipitate with povidone in a weight ratio of 1:1 prepared in Example 6.

FIG. 7 is scanning electron microscope image of an amorphous aprepitant coprecipitate with povidone in a weight ratio of 1:1 prepared in Example 6.

FIG. 8 is a scanning electron microscope image of powder prepared in Control Example 1.

### DETAILED DESCRIPTION OF THE INVENTION

The term “coprecipitate” as used herein refers to compositions comprising amorphous aprepitant together with at least one pharmaceutically acceptable carrier, being prepared by removing solvent from a solution containing both of them.

The term “pharmaceutical composition” as used herein refers to pharmaceutical formulations comprising amorphous aprepitant coprecipitates as described below along with one or more additional pharmaceutically acceptable excipients, as required to formulate the coprecipitates of amorphous aprepitant into pharmaceutical compositions for the delivery of aprepitant.

In one aspect, the invention provides pharmaceutical compositions of amorphous aprepitant coprecipitates together with at least one pharmaceutically acceptable excipient.

The amorphous aprepitant coprecipitate with a pharmaceutically acceptable carrier obtained using the disclosed process is characterized by any of their X-ray diffraction (“XRD”) pattern, infrared absorption (“IR”) spectrum, and differential scanning calorimetry (“DSC”) curve.

Amorphous coprecipitates of aprepitant with a pharmaceutically acceptable carrier can be characterized by their XRD patterns. All XRD data reported herein were obtained using Cu Kα-1 radiation, having the wavelength 1.541 Å, and were obtained using a Bruker Axe D8 Advance Powder X-ray Diffractometer. The XRD patterns of the drawing figures have a vertical axis of intensity units and a horizontal axis which is the 2θ angle, in degrees.

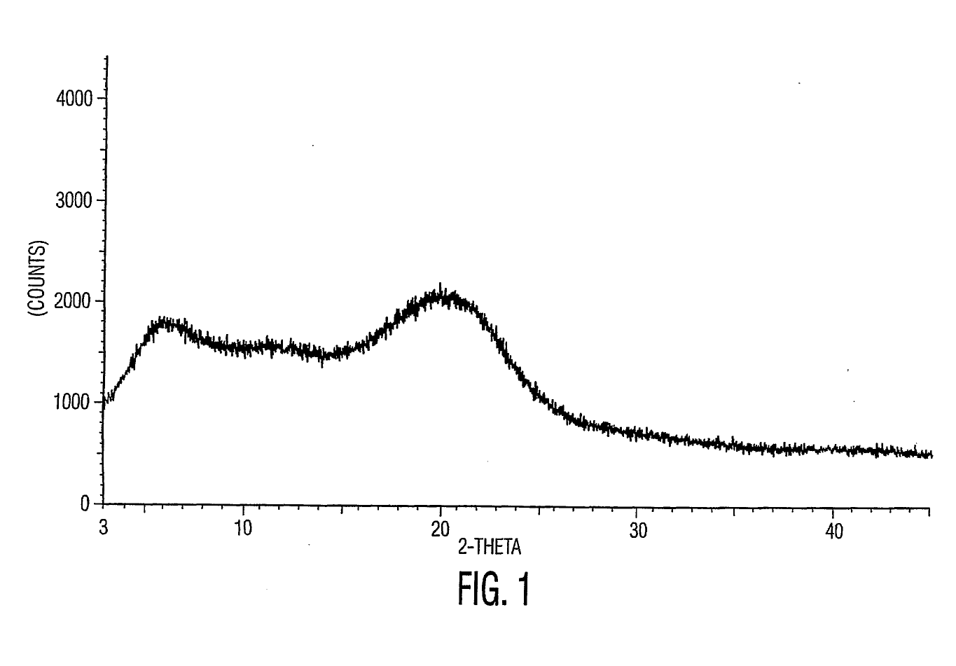

Amorphous aprepitant and amorphous combinations of aprepitant with pharmaceutically acceptable carriers are characterized by their XRD patterns showing a plain halo with no well-defined peaks, which is characteristic of an amorphous solid, as shown in FIGS. 1-4.

The IR spectra of an amorphous combination of aprepitant with pharmaceutically acceptable carriers has been recorded on a Perkin Elmer System Spectrum I model spectrophotometer, between 450 cm⁻¹ and 4000 cm⁻¹, with a resolution of 4 cm⁻¹ in a potassium bromide pellet, the test compound being at a concentration of 1% by mass.

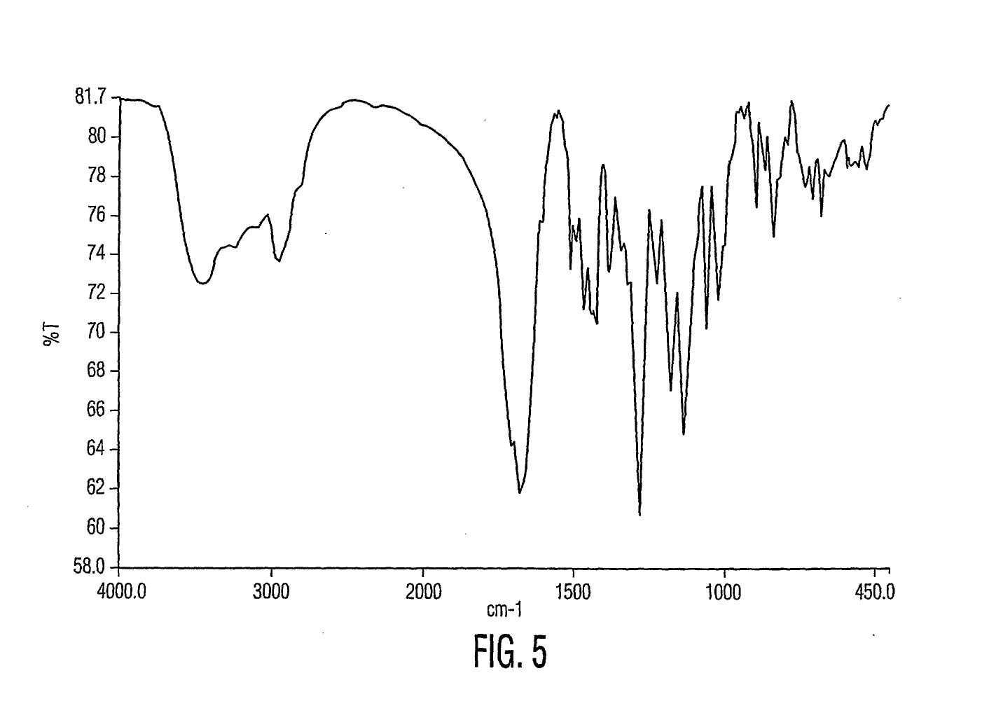

Amorphous aprepitant coprecipitate with povidone is characterized by an infrared absorption spectrum in potassium bromide having peaks at about 682, 708, 840, 898, 1060, 1022, 1131, 1280, 1463, 1711, 2952, and 3437, ±5 cm⁻¹. Amorphous aprepitant coprecipitate with povidone is also characterized by its infrared absorption spectrum in potassium bromide substantially in accordance with the spectrum of FIG. 5.

Differential scanning calorimetric analysis was carried out in a DSC Q1000 model instrument from TA Instruments with a ramp of 5° C./minute with a modulation time of 60 seconds and a modulation temperature of ±1° C. The starting temperature was 0° C. and ending temperature was 200° C.

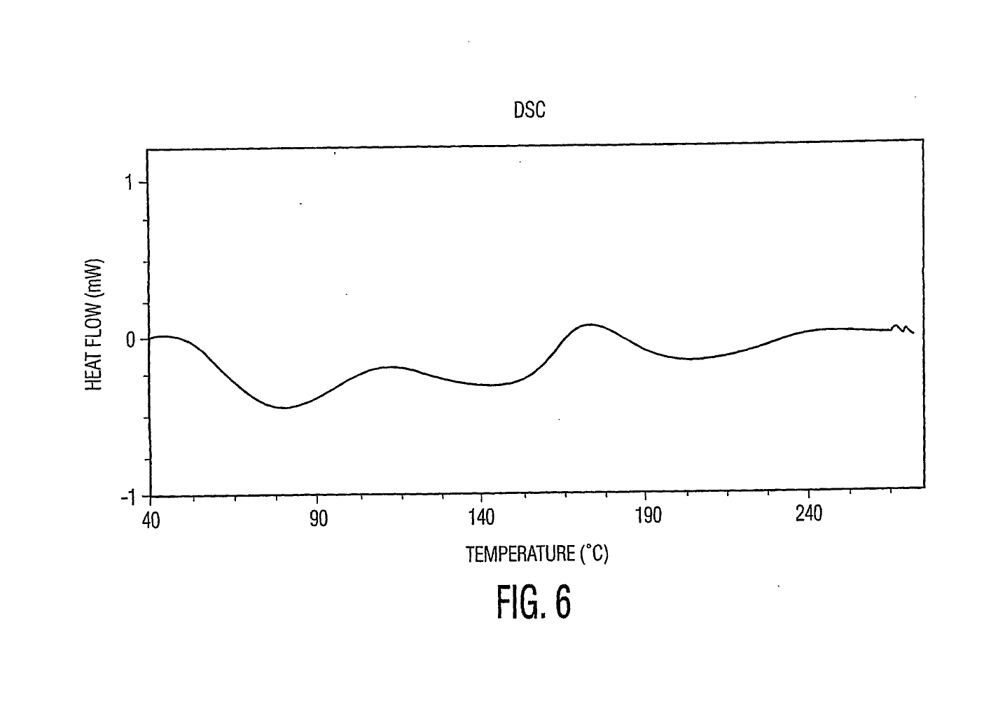

The amorphous aprepitant coprecipitate with povidone in a 1:1 weight ratio has a characteristic differential scanning calorimetry curve substantially in accordance the curve of with FIG. 6. The amorphous aprepitant coprecipitate with povidone in a 1:1 weight ratio also has a characteristic differential scanning calorimetry curve having an onset of the glass transition at about 53° C., a half point glass transition at about 79° C. and ending of glass transition at about 115° C.

In another aspect, the invention provides a process for the preparation of coprecipitates of amorphous aprepitant with a pharmaceutically acceptable carrier.

In an embodiment, the process for preparation of coprecipitates of amorphous aprepitant with a pharmaceutically acceptable carrier comprises the steps of:

a) providing a solution of aprepitant and a pharmaceutically acceptable carrier;

b) removing the solvent; and

c) optionally, drying the solid obtained.

Step a) involves providing a solution of aprepitant and a pharmaceutically acceptable carrier.

The solution of aprepitant may be obtained by dissolving aprepitant in a suitable solvent, or such a solution may be obtained directly from a reaction in which aprepitant is formed.

When the solution is prepared by dissolving aprepitant in a suitable solvent, any form of aprepitant such as any crystalline form of aprepitant, including any solvates and hydrates, may be utilized for preparing the solution.

The pharmaceutical carrier can be dissolved in a solution containing aprepitant, or aprepitant can be dissolved in a solution containing a pharmaceutical carrier. Alternatively, a solution containing aprepitant can be combined with a solution containing a pharmaceutical carrier, and the solvents used for preparing the different solutions need not be the same as long as the solvents have mutual solubility and form a single phase. In any event, the aprepitant must be completely soluble in the solvents used and should provide a clear solution. The presence of undissolved crystals could lead to the formation of a material that is not completely amorphous.

Suitable solvents that can be used for dissolving aprepitant either alone or along with a pharmaceutically acceptable carrier include, but are not limited to: alcohols such as methanol, ethanol, isopropyl alcohol, n-propanol, and the like; halogenated hydrocarbons such as dichloromethane, 1,2-dichloroethane, chloroform, carbon tetrachloride and the like; ketones such as acetone, ethyl methyl ketone, methyl isobutyl ketone and the like; esters such as ethyl acetate, n-propyl acetate, n-butyl acetate, t-butyl acetate and the like; ethers such as diethyl ether, dimethyl ether, diisopropyl ether; hydrocarbons such as toluene, xylene, n-heptane, cyclohexane, n-hexane and the like; nitriles such as acetonitrile, propionitrile and the like; or mixtures thereof.

The pharmaceutically acceptable carriers that can be used for the preparation of coprecipitates with amorphous aprepitant include, but are not limited to, pharmaceutical hydrophilic carriers such as polyvinylpyrrolidone (homopolymers, also called “povidone,” or copolymers of N-vinylpyrrolidone), gums, cellulose derivatives (including hydroxypropyl methylcellulose, hydroxypropyl cellulose and others), cyclodextrins, gelatins, hypromellose phthalate, sugars, polyhydric alcohols. The use of mixtures of more than one of the pharmaceutical carriers to provide desired release profiles or for the enhancement of stability is within the scope of this invention. Also, all viscosity grades, molecular weights, commercially available products, their copolymers, mixtures are all within the scope of this invention without limitation.

These lists of solvents and pharmaceutically acceptable carriers are merely representative of those that can be used, and the lists are not intended to be exhaustive or limiting. Generally, the more volatile solvents are preferred to reduce the energy requirements for subsequent solvent removal.

The dissolution temperatures can range from about 20 to 120° C. depending on the solvent used for dissolution. Any other temperature is also acceptable as long as a clear solution of aprepitant either alone or together with a pharmaceutically acceptable carrier is provided.

The quantity of solvent used for dissolution depends on the solvent and the dissolution temperature adopted. The concentration of aprepitant in the solution may generally range from about 0.1 to about 10 g/ml in the solvent. In general, the volumes will be kept to a minimum, to facilitate the eventual solvent removal.

The solution can optionally be treated with materials such as carbon for clarification or with sodium sulfate for moisture removal.

Optionally, the solution obtained above can be treated to remove the undissolved particles, prior to further process steps. Any undissolved particles can be removed suitably by filtration, centrifugation, decantation, and other techniques. The solution can be filtered by passing through paper, glass fiber, or other membrane material, or a particulate filtration medium such as celite or flux calcined diatomaceous earth (Hyflow). Depending upon the equipment used and the solution properties, such as concentration and temperature of the solution, the filtration apparatus may need to be preheated to avoid crystallization.

Step b) involves removal of the solvent from the solution obtained from step a), using a suitable technique.

Removal of the solvent may be carried out suitably using techniques such as evaporation, atmospheric distillation, or distillation under vacuum.

Distillation of the solvent may be conducted under a vacuum such as below about 100 mm Hg to below about 600 mm Hg, and at elevated temperatures such as about 20° C. to about 70° C. Any temperature and vacuum conditions can be used as long as there is no increase in the impurity levels of the product due to decomposition, etc.

Suitable techniques which can be used for the distillation include, without limitation thereto, distillation using a rotational evaporator device such as a Buchi Rotavapor, spray drying, agitated thin film drying (“ATFD”), and the like.

Techniques such as Buchi Rotavapor evaporation and distillation under vacuum may be suitable for laboratory-scale processes such as for quantities up to about 100 g. Other techniques such as spray drying and ATFD are more suitable for industrial scale production with a batch size of at least about 100 g or about 1 kg, or greater. Generally, techniques providing a rapid solvent removal will be utilized to provide the desired amorphous form of aprepitant.

The amorphous material obtained from step b) can be collected from the equipment using techniques such as by scraping the container. Other product collection techniques will be used for spray drying, and are well known in the art.

Step c) involves an optional drying of the product obtained from step b) to afford the amorphous aprepitant coprecipitate with a pharmaceutically acceptable carrier, substantially free of residual solvents.

Drying can be carried out under reduced pressure until the residual solvent content reduces to the desired amount, such as an amount that is within the limits given by the International Conference on Harmonization of Technical Requirements for Registration of Pharmaceuticals for Human Use (“ICH”) guidelines. The guideline solvent level depends on the type of solvent but is not more than about 5000 ppm, or about 4000 ppm, or about 3000 ppm.

The drying can be carried out at atomospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used, and the foregoing should be considered as only a general guidance.

Drying can be suitably carried out in a tray dryer, vacuum oven, air oven, or using a fluidized bed drier, spin flash dryer, flash dryer and the like. Drying equipment selection is well within the ordinary skill in the art.

The dried product can optionally be milled to get desired particle sizes. Milling or micronization can be performed prior to drying, or after the completion of drying of the product. The milling operation reduces the size of particles and increases surface area of particles. Drying is more efficient when the particle size of the material is smaller and the surface area is higher, hence milling will frequently be performed prior to the drying operation.

Milling can be done suitably using jet milling equipment like an air jet mill, or using other conventional milling equipment.

An amorphous coprecipitate of aprepitant with a pharmaceutically acceptable carrier obtained according to the process described in the present invention has solubility greater than that of the crystalline Form I or Form II of aprepitant. The aqueous solubility is up to about 60 times higher than crystalline Form I of aprepitant, and up to about 10 times higher than crystalline Form II of aprepitant. The coprecipitates generally have a solubility in water at room temperature at least about 0.25 mg/ml, or at least about 0.5 mg/ml, or at least about 1 mg/ml.

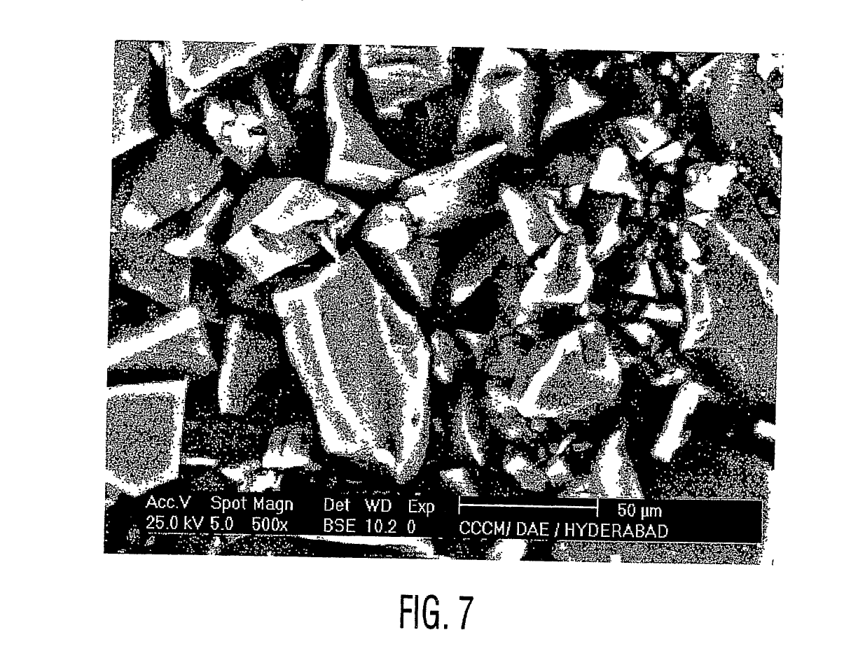

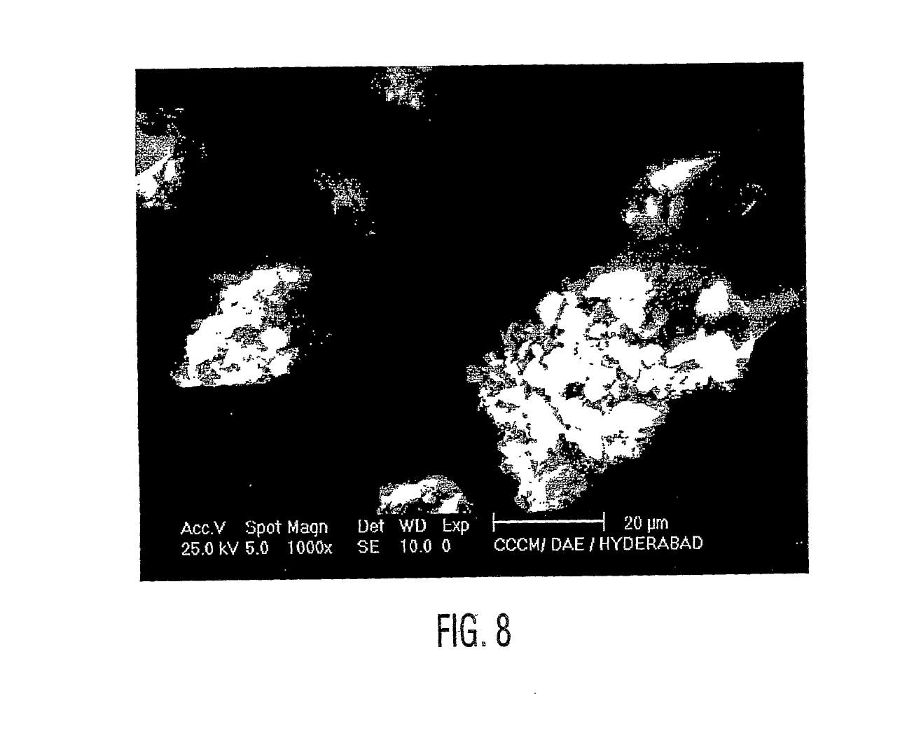

The amorphous coprecipitates of aprepitant with the pharmaceutically acceptable carrier obtained by the present process are a random distribution of the amorphous aprepitant and the pharmaceutically acceptable carrier in a particle matrix. While the invention should not be constrained by any particular theory, the coprecipitates have the characteristics of solid dispersions at a molecular level, being in the nature of solid solutions. The solid solutions, or molecular dispersions, provide homogeneous particles in which no discrete areas of only amorphous aprepitant and only pharmaceutically acceptable carrier can be observed. A scanning electron microscope study of aprepitant powder compositions has been performed using a Philips scanning electron microscope. The samples were prepared using carbon film. Powder coprecipitates of the present invention showed morphology under the scanning electron microscope having a large glassy particle nature, an image being shown as FIG. 7, and the material appears more homogenous than is the case when aprepitant and povidone are merely in intimate mixture. Material prepared using the Control Example 1 described below showed morphology under a scanning electron microscope that is independent particles having a deposition thereon of smaller particles, the image being shown as FIG. 8. These particles are heterogeneous, as aprepitant and povidone are not in an intimate mixture.

Amorphous combination of aprepitant with pharmaceutically acceptable carriers obtained in this invention contain less than about 5000 ppm, or less than about 3000 ppm, or less than about 1000 ppm of methanol, and less than about 200 ppm, or less than about 100 ppm of individual residual organic solvents.

Still another aspect of the invention provides an amorphous combination of aprepitant with a pharmaceutically acceptable carrier having a mean particle size less than about 100 μm and a bulk density of about 0.5 to 0.2 g/ml.

The D₁₀, D₅₀ and D₉₀ values are useful ways for indicating a particle size distribution. D₉₀ refers to the value for the particle size for which at least 90 volume percent of the particles have a size smaller than the value. Likewise D₅₀ and D₁₀ refer to the values for the particle size for which 50 volume percent and 10 volume percent of the particles have a size smaller than the value. Methods for determining D₁₀, D₅₀ and D₉₀ include laser diffraction, such as using Malvern Instruments Ltd. (of Malvern, Worcestershire, United Kingdom) equipment. The D₅₀ value can be considered the “mean” particle size.

In an embodiment, amorphous aprepitant and amorphous combination of aprepitant with pharmaceutically acceptable salts according to the invention have a mean particle size of less than about 100 μm, D₁₀ less than 10 μm or less than 5 μm, D₅₀ less than 50 μm or less than 40 μm, and D₉₀ less than 400 μm or less than 300 μm. There is no specific lower limit for any of the D values.

Bulk density used herein is defined as a ratio of apparent volume to mass of the material taken, called untapped bulk density, and also ratio of tapped volume to mass of material taken, called tapped bulk density. A useful procedure for measuring these bulk densities is described in United States Pharmacopeia 24, Test 616 (Bulk Density and Tapped Density), United States Pharmaceopeial Convention, Inc., 2000. The amorphous combinations of aprepitant with pharmaceutically acceptable carriers obtained according to the process described in this invention have a bulk density of 0.1 g/ml to 0.3 g/ml, or 0.1 g/ml to 0.2 g/ml, before tapping, and bulk density of 0.2 to 0.5 g/ml, or 0.2 to 0.3 g/ml, after tapping.

These powder coprecipitates have improved solubility properties and hence also have improved bioavailability characteristics.

The powder compositions of this invention as described in the different embodiments above are useful in the preparation of pharmaceutical compositions for the delivery of aprepitant. As used herein, pharmaceutical composition means a composition (medicament) for use in treating a mammal that includes aprepitant and is prepared in a manner that is appropriate for administration to a mammal, such as a human. A pharmaceutical composition contains one or more pharmaceutically acceptable excipients that are non-toxic to the mammal intended to be treated when the composition is administered in an amount effective to treat the mammal.

The pharmaceutical compositions may be in the form of encapsulated free flowing powders or granules; compressed solid dosage forms such as tablets like chewable or dispersible or mouth dissolving; pellets (extruded or fluidized) or beads or spheres or cores (water-soluble or insoluble or both) filled into sachets or capsules; enterable solutions, syrup, suspensions or dispersions; emulsions like micro-emulsions or multiple-emulsions; elixirs, troches, lozenges, lyophilized powders and the like.

Also the lyophilized powders or enteric solutions or suspensions or dispersions, emulsions like micro-emulsions or multiple-emulsions, of aprepitant can further be filled into hard or soft gelatin capsules.

The pharmaceutical compositions of the present invention may contain one or more diluents to make up the final composition mass so that it becomes easier for the patient and the caregiver to handle.

Common diluents that can be used in pharmaceutical formulations comprise microcrystalline cellulose (MCC), silicified MCC (e.g. Prosolv™ HD 90), micro fine cellulose, lactose, starch, pregelatinized starch, sugar, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide, and the like.

The pharmaceutical compositions may further include a disintegrant. Useful disintegrants include but not limited to methyl cellulose, microcrystalline cellulose, carboxymethyl cellulose calcium, carboxymethyl cellulose sodium (e.g. Ac-Di-Sol®, Primellose®), crospovidone (e.g. Kollidon®, Polyplasdone®), povidone K-30, guar gum, magnesium aluminum silicate, colloidal silicon dioxide (Aerosil®), polacrilin potassium, starch, pregelatinized starch, sodium starch glycolate (e.g. Explotab®), and sodium alginate.

Pharmaceutical compositions may further include ingredients such as, but are not limited to, pharmaceutically acceptable glidants, lubricants, opacifiers, colorants, sweeteners, thickeners and other commonly used excipients.

In an embodiment, the pharmaceutical compositions of the present invention are manufactured as described below. Granules of actives are prepared by sifting the actives and excipients through the desired mesh size sieve and then are mixed, such as by using a rapid mixer granulator or planetary mixer or mass mixer or ribbon mixer or fluid bed processor or any other suitable device. The blend can be granulated, such as by adding a solution of a binder whether alcoholic or hydro-alcoholic or aqueous in a low or high shear mixer, fluidized bed granulator, and the like, or by dry granulation. The granulate can be dried using a tray drier or fluid bed drier or rotary cone vacuum drier and the like. The sizing of the granules can be done using an oscillating granulator or comminuting mill or any other conventional equipment equipped with a suitable screen. Alternatively, granules can be prepared by extrusion and spheronization or roller compaction. The dried granulate particles are sieved, and then mixed with lubricants and disintegrants and compressed into a tablets.

Alternatively the manufacture of granules of actives can be performed by mixing with the directly compressible excipients or by roller compaction. The blend so obtained is compressed using a suitable device, such as a multi-station rotary machine to form slugs, which are passed through a multimill or may be subjected to any methods of particle size reduction starting known to a person skilled in the art, such as but not limited to fluid energy milling or ball milling or colloidal milling or roller milling or hammer milling and the like, equipped with a suitable screen. The milled slugs of actives are then lubricated and compressed into tablets.

In another embodiment, the improved bioavailable pharmaceutical compositions of the present invention are used in the treatment of disorders such as emesis induced by radiation, including radiation therapy such as in the treatment of cancer, post-operative nausea, and vomiting.

In yet another embodiment, the improved bioavailable pharmaceutical compositions of the present invention may be formulated optionally with one or more therapeutic agents for the treatment and relief of emesis or post-operative nausea and vomiting, such as 5-HT3 antagonist like ondansetron or granisetron or tropisetron, dopamine antagonists such as metoclopramide or domperidone, and GABA-B receptor agonists such as baclofen and the like.

### CONTROL EXAMPLE 1

### Preparation of Aprepitant and Povidone Mixture

8 grams of povidone was dissolved in 20 ml of water, 8 grams of aprepitant Form 1 was added to the above povidone-water solution and heated to 65-70° C. for 45 minutes. Solution was cooled to achieve a temperature between 0-5° C., stirred for 2 hours and then was filtered to separate the solid.

### CONTROL EXAMPLE 2

### Preparation of Amorphous Aprepitant

35 grams of aprepitant was dissolved in 300 ml of tetrahydrofuran to get a clear solution. This solution was spray dried using a spray drier (Jay Instruments & Systems Pvt. Ltd. India, Model LSD-348-PLC) maintaining feed rate at 110 ml per hour, aspiration rate at >1600 RPM to maintain negative pressure of 110-130 mm water, inlet temperature at 140° C., outlet temperature at 80° C. and atomization air pressure at 2.2 kg/cm². 20 grams of dried substance was collected.

The XRD pattern of the sample demonstrates the amorphous nature as shown in FIG. 1.

The following examples will further illustrate certain specific aspects and embodiments of the invention in greater detail and are not intended to limit the scope of the invention.

### EXAMPLE 1

### Coprecipitate of Aprepitant with Povidone in a Ratio of (1:1) Using Dichloromethane as the Solvent

1 gram of aprepitant and 1 gram of povidone (PVP K30) were dissolved in 200 ml of dichloromethane with the aid of heating to a temperature of 40° C. The solution was filtered in the hot condition and the dichloromethane was removed using distillation in a Buchi Rotavapor apparatus under a vacuum of 0-20 torr. 1.8 grams of a dried coprecipitate of aprepitant with povidone was obtained.

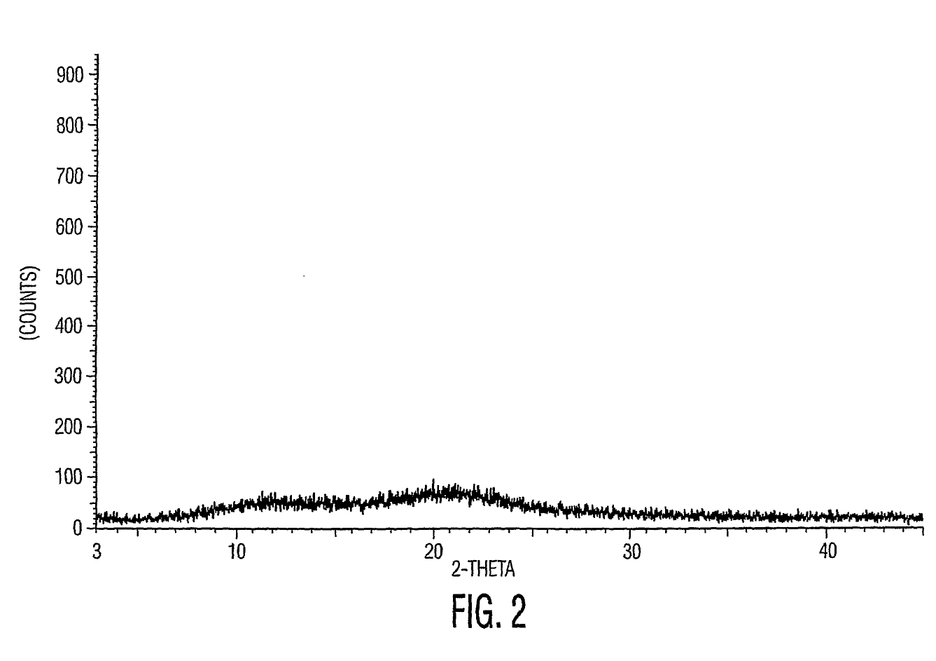

FIG. 2 is the XRD pattern of the product, demonstrating the amorphous nature of the coprecipitate.

### EXAMPLE 2

### Coprecipitate of Aprepitant with Povidone in a Ratio of (1:1) Using a Combination of Dichloromethane and Methanol as the Solvent

5 grams each of aprepitant and povidone (PVP K 30, average molecular weight approximately 50,000) were dispersed in 1000 ml of dichloromethane and stirred for 30 minutes. 20 ml of methanol was added and stirred for 5 minutes to get a clear solution. The solution was filtered using a Hyflow (flux calcined diatomaceous earth) bed and the solvents were removed using distillation in a Buchi Rotavapor apparatus under a vacuum of 0-22 torr. 10 grams of a dried coprecipitate of aprepitant with povidone were obtained.

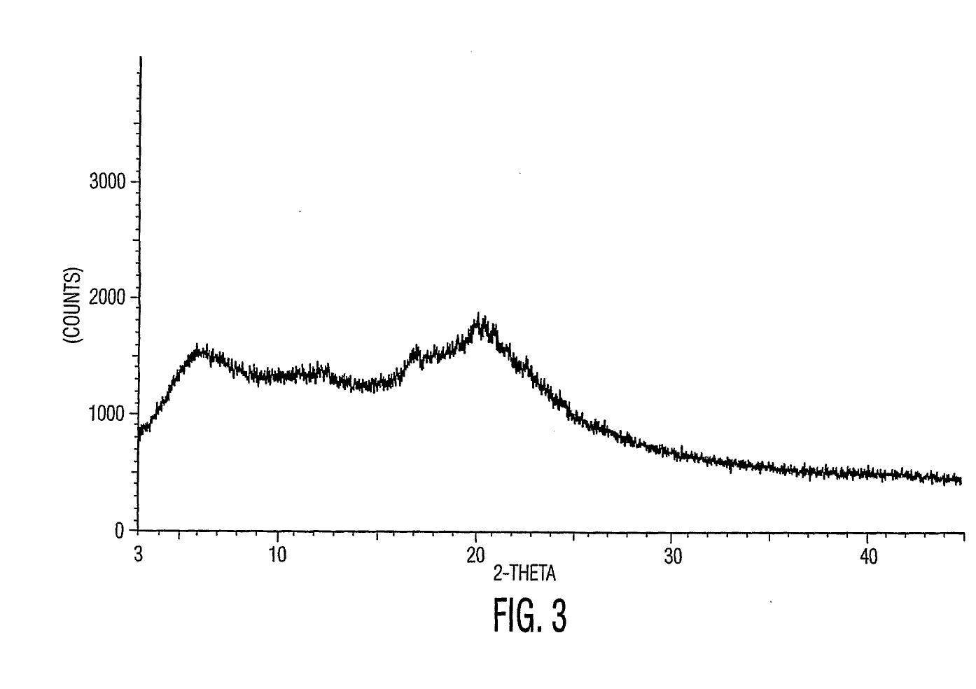

FIG. 3 is the XRD pattern of the product, demonstrating the amorphous nature of the coprecipitate.

### EXAMPLE 3

### Coprecipitate of Aprepitant with Povidone in a Ratio of (3:1) Using Dichloromethane as Solvent

1.5 gram of aprepitant and 0.5 gram of povidone (PVP K 30) were dissolved in 300 ml of dichloromethane with the aid of heating to a temperature of 40° C. The solution was filtered and the dichloromethane was removed using distillation is a Buchi Rotavapor apparatus under a vacuum of 2-20 torr. 1.8 grams of a dried coprecipitate of aprepitant with povidone was obtained.

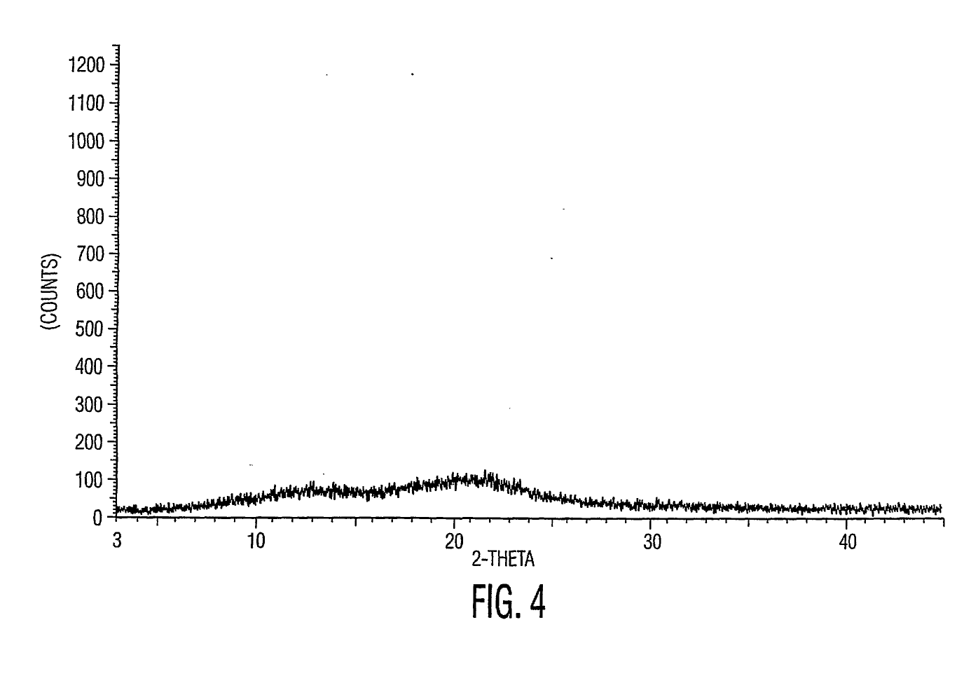

FIG. 4 is the XRD pattern of the product, demonstrating the amorphous nature of the coprecipitate.

### EXAMPLE 4

### Solubility Comparison

Solubility studies of different aprepitant powders were conducted. Sufficient aprepitant having either crystalline Form I or II, or in the form of an amorphous povidone coprecipitate, was added to a 25 ml test tube containing 10 ml of water at room temperature to get a saturated solution. The solutions were filtered and filtrate was used to obtain the absorbance at 210 nm. Absorbance was compared to standards prepared by dissolving aprepitant in methanol, then diluting aliquots with water.

Solubility of crystalline Form I in water was found to be 0.02 mg/ml, crystalline Form II in water was found to be 0.14 mg/ml and the coprecipitate obtained from Example 1 was found to be 1.1 mg/ml at room temperature.

These data demonstrate a significant enhancement in the solubility properties of the amorphous aprepitant coprecipitate when compared to either of the crystalline forms of aprepitant.

### EXAMPLE 5

### Capsule Composition of a Coprecipitate of Amorphous Aprepitant with Povidone

The coprecipitate of aprepitant with povidone prepared in Example 1 equivalent to 80 g aprepitant is sifted through a 40 mesh ASTM sieve and is mixed with pre-sifted 80 g of sucrose, 120 g of microcrystalline cellulose and 10 g of sodium starch glycolate and blended with 5 g of magnesium stearate and 5 g of talc. The blend is filled into hard gelatin capsules.

### EXAMPLE 6

### Preparation of Coprecipitate of Amorphous Aprepitant with Povidone in 1:1 Ratio

10 g of aprepitant and 10 g of povidone were taken into a round bottom flask and 2000 ml of dichloromethane was added to it. The mass was stirred for 30 minutes at 25° C. 10 ml of methanol was added to it and stirring was continued for another 20 minutes. The mass was filtered through a celite bed and the filtrate was taken into a Rotavapor flask. The filtrate was distilled on a Buchi Rotavapor at a temperature of 45° C. and pressure of 300 mm Hg to yield 19 g of the title coprecipitate.

### EXAMPLE 7

### Preparation of Coprecipitate of Amorphous Aprepitant with Povidone in 6:4 Ratio

3 g of aprepitant and 2 g of povidone were taken into a round bottom flask and 100 ml of dichloromethane was added to it. The mass was stirred for 30 minutes at 27° C. 3.0 ml of methanol was added to it and stirring was continued for another 20 minutes. The mass was filtered through a celite bed and the filtrate was taken into a Rotavapor flask. The filtrate was distilled on a Rotavapor at a temperature of 45° C. and pressure of 300 mm Hg to yield 4.7 g of the title coprecipitate.

### EXAMPLE 8

### Preparation of Coprecipitate of Amorphous Aprepitant with Povidone in 7:3 Ratio

3.5 g of aprepitant and 1.5 g of povidone were taken into a round bottom flask and 140 ml of dichloromethane was added to it. The mass was stirred for 25 minutes at 27° C. 3.5 ml of methanol was added and stirring was continued for another 20 minutes. The reaction mass was filtered over a celite bed and the filtrate was taken into a Rotavapor flask. The filtrate was distilled on a Rotavapor at a temperature of 45° C. and pressure of 300 mm Hg to yield 4.85 g of the title coprecipitate.

### EXAMPLE 9

### Preparation of Coprecipitate of Amorphous Aprepitant with Povidone in 6:4 Ratio

4.0 g of aprepitant and 1.0 g of povidone were taken into a round bottom flask and 180 ml of dichloromethane was added to it. The mass was stirred for 35 minutes at 27° C. 4.0 ml of methanol was added to it and stirring was continued for another 30 minutes. The mass was filtered through a celite bed and the filtrate was taken into a Rotavapor flask. The filtrate was distilled on a Buchi Rotavapor at a temperature of 45° C. and pressure of 300 mm Hg to yield 4.8 g of the title coprecipitate.

### EXAMPLE 10

### Storage Stability of Amorphous Aprepitant Coprecipitate with Povidone

Amorphous aprepitant coprecipitate with povidone prepared according to Example 2 was packaged in a sealed polyethylene bag and the bag was placed into a second bag along with a silica gel desiccant pouch and the second bag was sealed, then the package was stored at 40° C. and 75% relative humidity and checked for stability of the polymorphic form at intervals. After 15 and 30 days of storage, XRD patterns of samples were the same as the original pattern, indicating that the amorphous form had not changed. The appearance of the samples also remained unchanged.

1. A coprecipitate comprising amorphous aprepitant and a pharmaceutically acceptable carrier.

2. The coprecipitate of claim 1, which is in the form of a molecular dispersion.

3. The coprecipitate of either of claims 1, wherein a pharmaceutically acceptable carrier comprises polyvinylpyrrolidone.

4. The coprecipitate of claim 1, prepared by removing solvent from a solution comprising aprepitant and a pharmaceutically acceptable carrier.

5. The coprecipitate of claim 1, prepared by removing solvent from a solution comprising aprepitant and a pharmaceutically acceptable carrier, under reduced pressure.

6. The coprecipitate of claim 1, having an aqueous solubility at least about 0.25 mg/ml.

7. The coprecipitate of claim 1, having an aqueous solubility at least about 0.5 mg/ml.

8. The coprecipitate of claim 1, having an aqueous solubility at least about 1 mg/ml.

9. The coprecipitate of claim 3, having an aqueous solubility at least about 1 mg/ml.

10. The coprecipitate of claim 1, in which no discrete zones of amorphous aprepitant and of pharmaceutically acceptable carrier can be observed in a particle.

11. A process for preparing a coprecipitate of amorphous aprepitant and a pharmaceutically acceptable carrier, comprising:
a) providing a solution containing aprepitant and a pharmaceutically acceptable carrier;
b) removing solvent to form a solid; and
c) optionally, drying a formed solid.

12. The process of claim 11, wherein solvent is removed in b) at temperatures about 20° C. to about 70° C.

13. The process of claim 11, wherein solvent is removed in b) under a vacuum.

14. The process of claim 11, wherein solvent is removed in b) using a technique comprising spray drying or agitated thin film drying.

15. A coprecipitate of amorphous aprepitant and a pharmaceutically acceptable carrier, prepared by a process of claim 11.

16. A coprecipitate comprising amorphous aprepitant and polyvinylpyrrolidone, in the form of a molecular dispersion.

17. The coprecipitate of claim 16, having an aqueous solubility at least about 0.25 mg/ml.

18. The coprecipitate of claim 16, having an aqueous solubility at least about 0.5 mg/ml.

19. The coprecipitate of claim 16, having an aqueous solubility at least about 1 mg/ml.

20. The coprecipitate of claim 16, in which no discrete zones of amorphous aprepitant and of polyvinylpyrrolidone can be observed in a particle.

21. A coprecipitate of amorphous aprepitant and a pharmaceutically acceptable carrier, prepared by a process of claim 12.

22. A coprecipitate of amorphous aprepitant and a pharmaceutically acceptable carrier, prepared by a process of claim 13.

23. A coprecipitate of amorphous aprepitant and a pharmaceutically acceptable carrier, prepared by a process of claim 14.
